(19) United States
(12) Patent Application Publication
(10) Pub. No.: US 2002/0144302 A1
(43) Pub. Date: Oct. 3, 2002

(54) NOVEL CONSTRUCTS AND VECTORS FOR THE TARGETED AND INDUCIBLE EXPRESSION OF GENES

(76) Inventors: ABDERRAHIM MAHFOUDI, PASTOUREAUX (FR); PATRICK BENOIT, PARIS (FR); DIDIER BRANELLEC, SAINT-HILAIRE (FR); PATRICE DENEFLE, SAINT MAUR (FR); NICOLAS DUVERGER, PARIS (FR); LAURENCE BERTHOU, PARIS (FR); JOHAN AUWERX, MILLONFOSSE (FR); BART STAELS, KRAAINEM (BE)

Correspondence Address: WILEY, REIN & FIELDING, LLP, ATTN: PATENT ADMINISTRATION, 1776 K. STREET N.W., WASHINGTON, DC, 20006, US

(21) Appl. No.: 09/297,910

(22) PCT Filed: Nov. 6, 1997
(86) PCT No.: PCT/FR97/01992

(30) Foreign Application Priority Data
Nov. 8, 1996 (FR) 96/13691

(51) Int. Cl. A61K 38/17; C12N 15/86; C07H 21/04

(52) U.S. Cl. 800/21; 435/456; 435/320.1; 536/23.1; 514/2

(57) ABSTRACT

The invention concerns novel constructs and novel vectors for the targeted and inducible expression of genes. It describes in particular novel hybrid promoters and their use for the expression of genes in hepatic cells, in vitro, ex vivo or in vivo.

The present invention relates to the field of biology, and in particular the field of regulation of the expression of genes. It describes in particular new constructs and new vectors which allow a targeted and inducible expression of genes. The present invention can be used in numerous fields, and in particular for the production of recombinant proteins, for the creation of transgenic animal models, for the creation of cell lines, for the development of screening tests, or in gene or cell therapy.

The possibility of controlling and directing the expression of genes constitutes a very important factor in the development of biotechnologies. In vitro, it makes it possible to improve the conditions for producing recombinant proteins, by decoupling, for example, the cellular growth phase and the production phase. Still in vitro, it also makes it possible to create cell lines capable of producing certain molecules at selected periods. Thus, it is feasible to construct cell lines producing, in a regulated manner, proteins which transcomplement defective viral genomes. Still in vitro, a regulated system of expression allows the development of tests for screening molecules which act on the control of the expression of genes. The control of the expression of genes is also very important for therapeutic approaches ex vivo or in vivo, in which the possibility of selectively controlling the production of a therapeutic molecule is essential. Indeed, depending on the applications, depending on the gene to be transferred, it is important to be able to target certain tissues or only certain parts of an organism in order to concentrate the therapeutic effect and to limit dissemination and side effects.

This targeting may be achieved using vectors exhibiting a given cellular specificity. Another approach consists in using expression signals specific for certain cell types. In this regard, so-called specific promoters have been described in the literature, such as the promoter of the genes encoding pyruvate kinase, villin, GFAP, the fatty acid-binding intestinal protein promoter, the smooth muscle cell α-actin promoter, or the promoter of the human albumin gene for example. However, while these promoters exhibit a degree of tissue specificity, they are not regulatable and, as a result, offer limited possibilities of control. Other, more complex, systems have been described in the literature. Thus, application WO 96/01313 describes a system for the expression of genes which is regulated by tetracyclin. Likewise, Wang et al., (PNAS 91 (1994) 8180) have described a system for the expression of genes which is regulated by RU486. Evans et al. have, for their part, described a system based on the receptor for ecdysone, an insect hormone (PNAS 93 (1996) 3346). However, these various systems, although inducible, do not exhibit tissue specificity. As a result, they do not make it possible, on their own, to target the expression at the level of the desired organs or tissues, but simply to induce or repress expression in a ubiquitous manner. Furthermore, the tetracyclin system exhibits a relatively weak level of regulation, less than a factor of five. Moreover, these systems function with hybrid molecules and require the cotransfection of at least 2 constructs. In addition, they use heterologous elements and therefore risk generating immune reactions.

The invention now describes new constructs allowing the targeted and regulated expression of genes. The invention describes in particular recombinant vectors allowing expression of inducible and hepatospecific genes. The invention also describes new promoter constructs having improved levels of regulation. The present invention thus offers a particularly effective means for targeting the expression of genes in hepatic cells, in vivo or in vitro, and for regulating this expression.

The present application is based in particular on the use of the promoter for the human gene for apolipoprotein AII. Apolipoprotein AII (apoAII) is one of the major protein constituents of the high density lipoproteins (HDL). ApoAII is synthesized predominantly in the liver, although contradictory results suggest a synthesis also in the intestine. The human gene for apoAII has been cloned and sequenced (Tsao et al., J. Biol. Chem. 260 (1985) 15222). The promoter region extends over about 1 kb upstream of the codon for initiation of transcription. It comprises regulatory elements located at the level of nucleotides −903 to −680, as well as additional multiple sites situated at the level of the intermediate region (nucleotides −573 to −255) and the proximal region (−126 to −33). The optimum expression is obtained when the nuclear factors are bound to the proximal and distal regulatory elements of the promoter.

The sequence of the promoter of the human gene for apoAII, from residue 911 to +29, is represented on the sequence SEQ ID No. 1.

Contradictory mechanisms for the regulation of the apoAII promoter in man and in rodents have been observed. In one case, stimulation by fibrates was observed, in the other, an inhibition. Fibrates, often used as hypolipidaemic agents, belong to the chemical family of peroxisome proliferators, since they induce a hepatomegaly linked to the proliferation of peroxisomes in rodents. Their action is mediated by activated receptors (PPAR: “Peroxisome Proliferator Activated Receptor”), a group of 4 distinct nuclear receptors (α, β, γ, δ). The PPARs belong to the superfamily of nuclear hormone receptors which bind to specific response elements designated PPRE (“Peroxisome Proliferator Response Element”). PPREs have been identified in numerous genes encoding enzymes involved in the β-oxidation pathway, which have proved to be inducible by fibrates.

The applicant has now developed a system for the expression of hepatospecific genes inducible by fibrates, which can be used in vitro and in vivo. More particularly, the applicant has constructed, for the first time, a vector having tropism for the liver which allows the expression of genes selectively in the liver or the hepatic cells, and inducibly by fibrates. The applicant has also constructed new promoters derived from the promoter of the human apoAII gene, having improved inducibility and strength properties.

A first subject of the invention consists in a recombinant vector for the inducible and hepatospecific expression of a molecule, characterized in that it comprises an expression cassette consisting of a nucleic acid encoding the said molecule, placed under the control of the promoter of the human apolipoprotein AII gene.

According to a particularly preferred variant, the recombinant vector is a viral vector derived from adenoviruses, comprising, inserted into its genome, the said expression cassette.

In a particularly remarkable manner, the applicant has indeed shown that such an adenovirus made it possible to express a gene specifically in the liver, that this expression was strongly inducible in vivo by fibrates, and that the levels of expression obtained were comparable to those described previously with the strongest constitutive promoters.

The hepatospecific character of the viruses of the invention means that these viruses allow the expression of a gene in a very selective manner in hepatic cells, in vitro, ex vivo or in vivo. A weak nonspecific expression in other tissues or cell types can be tolerated, as long as a very predominant expression is observed in the hepatic cells (preferably more than 80% of the cells expressing the transgene are hepatic cells, still more preferably more than 90%). In particular, contrary to the contradictory indications noted in the prior art, the virus according to the invention does not induce any detectable expression in the intestine and therefore offers a particularly high selectivity. This is very important for approaches involving the transfer and expression of toxic genes, for which a very high level of selectivity is required. Moreover, as indicated above, inducible systems described in the prior art exhibit average inducibility, of a factor of about five. The results presented in the examples demonstrate that the adenovirus of the invention is inducible by a factor of about ten. The level of inducibility is also very important for obtaining a control of the quantity of molecules delivered in vivo. This is particularly sensitive in the case of immunogenic molecules or of molecules capable of generating inflammatory responses. This is also particularly important for the expression of molecules whose biological efficacy involves high concentrations. Moreover, another particularly remarkable characteristic of the vector of the invention lies in the high levels of expression obtained. Indeed, the inducible systems generally exhibit, as a corollary, average or even low levels of expression. Surprisingly and advantageously, the applicant has shown that the system of the invention makes it possible to obtain levels of expression in vivo which are comparable to those described for the strongest constitutive promoters. The system of the invention therefore combines, for the first time, remarkable properties of selectivity, inducibility and strength.

One of the features of the invention therefore lies in the use of the promoter of the human gene for apoAII. Another feature of the invention lies in the construction of vectors derived from adenoviruses. The vectors according to the invention combine remarkable properties of gene transfer, safety, tissue specificity, inducibility and strength.

Advantageously, the promoter used in the viruses of the invention comprises the regulatory elements of the promoter of the apoAII gene. More particularly, these elements are located at the level of nucleotides −903 to −680; −573 to −255; and −126 to −33 of the human gene for apoAII. In this regard, according to a specific variant, the promoter comprises residues −911 to +29 of the apoAII gene (sequence SEQ ID No. 1).

It is understood that shorter or longer forms of the promoter can be used. Thus, on the 3′ side, it is important that the promoter comprises the site for initiation of transcription of the apoAII gene (numbered +1 on SEQ ID No. 1). On the other hand, it is preferable that this promoter does not contain the first intron of the apoAII gene, which starts at nucleotide +38. Thus, advantageously, the fragment used possesses a 3′ end between residues +5 and +35, more preferably +10 and +30 of the apoAII gene. As for the 5′ end, it is preferable, in order to obtain high levels of expression in the liver, to conserve, at least in part, the site for binding of the hepatic factors. This site is located at the level of nucleotides −903 to −680. As a result, advantageously, the fragment used possesses a 5′ end located upstream of nucleotide −903. This end may be located, for example, in the −950 to −910 region. Moreover, for reasons to do with cloning capacity, it is advantageous to use a promoter region of reduced size. As a result, the use of a fragment whose 5′ end is located in the −925 to −910 region is preferred.

According to an advantageous variant of the invention, the promoter comprises the regulatory elements located at the level of nucleotides −903 to −680 (or −903 to −720) and −126 to −33, but not the intermediate elements located at the level of nucleotides −573 to −255. In particular, the promoter used advantageously comprises a deletion in the region between residues −710 and −150. By way of specific example, the promoter may advantageously consist of a variant of the sequence SEQ ID No. 1 obtained by deletion of residues 708−210. Still more preferably, the promoter used comprises a deletion in the region between residues −670 and −210. By way of specific example, the promoter may advantageously consist of a variant of the sequence SEQ ID No. 1 obtained by deletion of residues 653−210.

The results presented in the examples show indeed that this type of construct has a high strength and exhibits inducibility by fibrates greater than the native promoter of apoAII, in an adenoviral context. These constructs are therefore advantageous from the point of view of the regulatory properties, and from the point of view of the cloning capacity of the vector, since the promoter region is reduced.

According to another embodiment, the adenoviruses according to the invention comprise, as promoter, a variant of the promoter of the apolipoprotein AII gene comprising a repeat of J units. The multiplication of the J region makes it possible advantageously to also increase the inducible character of the promoter by fibrates. The J region consists of the sequence TCAACCTTTACCCTGGTAG (SEQ ID No. 2, underlined on SEQ ID No. 1). It is located in the AII promoter at the level of nucleotides −734 to −716 of the promoter. The applicant has now constructed recombinant viruses comprising promoters modified at the level of the J region. These viruses exhibit particularly advantageous properties for the transfer and expression of genes, in vitro and in vivo.

Preferably, the promoter comprises 2 to 5 J units. Still more preferably, it comprises 3 J units. For the construction of these variants, the repeat of J units may be positioned in 5′ of the promoter, in 3′ of the promoter, or inserted into the sequence of the promoter, preferably at the level of the native J sequence. Moreover, the multiplication of J units can be advantageously combined with a deletion as described above. This makes it possible to obtain a promoter having further improved properties in terms of power and control of the expression of genes.

The various constructs can be prepared according to molecular biology techniques known to persons skilled in the art. Thus, starting with the sequence SEQ ID No. 1, persons skilled in the art can carry out various deletions by selecting appropriate restriction enzymes. The deletions can also be carried out by site-directed mutagenesis or by PCR. Moreover, the J regions can be synthesized artificially by means of nucleotide synthesizers, and then inserted into or fused with the promoter by PCR or by cleavage and ligation by means of appropriate enzymes. These various approaches are illustrated in the examples.

As indicated above, the remarkable capacities of the vectors of the invention stem from the promoter used, and from the choice of the vector. The demonstration of the functionality of the promoters in an adenoviral context in vivo has indeed allowed the preparation of these highly performing vectors.

Adenoviruses are viruses with a linear double-stranded DNA having a size of about 36 (kilobases) kb. Various serotypes exist, whose structure and properties vary somewhat, but which exhibit a comparable genetic organization. More particularly, recombinant adenoviruses may be of human or animal origin. As regards the adenoviruses of human origin, there may be mentioned preferably those classified in group C, in particular the type 2 (Ad2), 5 (Ad5), 7 (Ad7) or 12 (Ad12) adenoviruses. Among the various adenoviruses of animal origin, there may be preferably mentioned the adenoviruses of canine origin, and in particular all the CAV2 adenovirus strains [Manhattan or A26/61 (ATCC VR-800) strain for example]. Other adenoviruses of animal origin are cited particularly in application WO 94/26914 incorporated into the present by reference.

The genome of adenoviruses comprises in particular an inverted terminal repeat (ITR) at each end, an encapsidation sequence (Psi), early genes and late genes. The main early genes are contained in the E1, E2, E3 and E4 regions. Among these, the genes contained in the E1 region in particular are necessary for viral propagation. The main late genes are contained in the L1 to L5 regions. The genome of the Ads adenovirus has been completely sequenced and is available on a database (see particularly Genebank M73260). Likewise, parts, or even all of other adenoviral genomes (Ad2, Ad7, Ad12 and the like) have also been sequenced.

For their use as recombinant vectors, various constructs derived from adenoviruses have been prepared, incorporating various therapeutic genes. In each of these constructs, the adenovirus was modified so as to make it incapable of replicating in the infected cell. Thus, the constructs described in the prior art are adenoviruses deleted off the E1 region, essential for viral replication, into which are inserted the heterologous DNA sequences (Levrero et al., Gene 101 (1991) 195; Gosh-Choudhury et al., Gene 50 (1986) 161). Moreover, to improve the properties of the vector, it has been proposed to create other deletions or modifications in the adenovirus genome. Thus, a heat-sensitive point mutation was introduced into the ts125 mutant, making it possible to inactivate the 72 kDa DNA-binding protein (DBP) (Van der Vliet et al., 1975). Other vectors comprise a deletion of another region essential for viral replication and/or propagation, the E4 region. The E4 region is indeed involved in the regulation of the expression of the late genes, in the stability of the late nuclear RNAs, in the extinction of the expression of the proteins of the host cell and in the efficiency of the replication of the viral DNA. Adenoviral vectors in which the E1 and E4 regions are deleted therefore possess a very reduced viral gene expression and transcriptional background noise. Such vectors have been described for example in applications WO 94/28152, WO 95/02697, WO 96/22378). In addition, vectors carrying a modification at the level of the IVa2 gene have also been described (WO 96/10088).

In a preferred embodiment of the invention, the recombinant adenovirus is a group C human adenovirus. More preferably, it is an Ad2 or Ad5 adenovirus.

Advantageously, the recombinant adenovirus used within the framework of the invention comprises a deletion in the E1 region of its genome. Still more particularly, it comprises a deletion in the E1a and E1b regions. By way of a precise example, there may be mentioned deletions affecting nucleotides 454-3328; 382-3446 or 357-4020 (with reference to the Ads genome).

According to a preferred variant, the recombinant adenovirus used within the framework of the invention comprises, in addition, a deletion in the E4 region of its genome. More particularly, the deletion in the E4 region affects all the open reading frames. There may be mentioned, by way of a precise example, the 33466-35535 or 33093-35535 deletions. Other types of deletions in the E4 region are described in applications WO 95/02697 and WO 96/22378, incorporated into the present by reference.

The expression cassette can be inserted into various sites of the recombinant genome. It can be inserted at the level of the E1, E3 or E4 region, as a replacement for the deleted or surplus sequences. It can also be inserted into any other site, outside the sequences necessary in cis for the production of the viruses (ITR sequences and encapsidation sequence).

The recombinant adenoviruses are produced in an encapsidation line, that is to say a cell line capable of complementing in trans one or more of the functions deficient in the recombinant adenoviral genome. One of these lines is for example the line 293 into which part of the adenovirus genome has been integrated. More precisely, the line 293 is a human kidney embryonic cell line containing the left end (about 11-12%) of the genome of the serotype 5 adenovirus (Ads), comprising the left ITR, the encapsidation region, the E1 region, including E1a and E1b, the region encoding protein pIX and part of the region encoding protein pIVa2. This line is capable of transcomplementing recombinant adenoviruses defective for the E1 region, that is to say lacking all or part of the E1 region, and of producing viral stocks having high titres. This line is also capable of producing, at a permissive temperature (32° C.), virus stocks comprising, in addition, the heat-sensitive E2 mutation. Other cell lines capable of complementing the E1 region have been described, based in particular on human lung carcinoma cells A549 (WO 94/28152) or on human retinoblasts (Hum. Gen. Ther. (1996) 215). Moreover, the lines capable of transcomplementing several adenovirus functions have also been described. In particular, there may be mentioned lines complementing the E1 and E4 regions (Yeh et al., J. Virol. 70 (1996) 559; Cancer Gen. Ther. 2 (1995) 322; Krougliak et al., Hum. Gen. Ther. 6 (1995) 1575) and lines complementing the E1 and E2 regions (WO 94/28152, WO 95/02697, WO 95/27071).

The recombinant adenoviruses are usually produced by introducing the viral DNA into the encapsidation line, followed by lysis of the cells after about 2 or 3 days (the kinetics of the adenoviral cycle being 24 to 36 hours). For carrying out the process, the viral DNA introduced may be the complete recombinant viral genome, optionally constructed in a bacterium (WO 96/25506) or in a yeast (WO 95/03400), transfected into the cells. It may also be a recombinant virus used to infect the encapsidation line. The viral DNA may also be introduced in the form of fragments each carrying part of the recombinant viral genome and a zone of homology which makes it possible, after introduction into the encapsidation cell, to reconstitute the recombinant viral genome by homologous recombination between the various fragments.

After lysis of the cells, the recombinant viral particles are isolated by caesium chloride gradient centrifugation. An alternative method has been described in application FR 96/08164 incorporated into the present by reference.

The recombinant vector having tropism for the liver can also be constructed using a plasmid-type non-viral vector, in particular as described in applications WO 96/26270 and PCT/FR96/01414.

As indicated above, the vectors of the invention allow the regulated production, at a high level, and the hepatospecific production of molecules of interest. The molecule of interest is advantageously a therapeutic molecule. It may be a protein or a nucleic acid (tRNA, antisense RNA, and the like).

In a particularly preferred manner, the therapeutic molecule is a protein secreted into the bloodstream. There may be mentioned, by way of example, enzymes, blood derivatives, hormones, lymphokins: interleukins, interferons, TNF and the like (FR 9,203,120), growth factors, neurotransmitters or precursors thereof or synthesis enzymes, trophic factors: BDNF, CNTF, NGF, IGF, GMF, aFGF, bFGF, NT3, NT5 and the like; apolipoproteins: ApoAI, ApoAIV, ApoE and the like (WO 94/25073), dystrophin or a minidystrophin (WO 93/06223), tumour suppressor genes: p53, Rb, Rap1A, DCC, k-rev and the like (WO 94/24297), genes encoding the factors involved in clotting: Factors VII, VIII, IX and the like, or alternatively all or part of a natural or artificial immunoglobulin (Fab, ScFv, etc., WO 94/29446).

To ensure the secretion of the protein, the expression cassette advantageously comprises an appropriate signal sequence. It may be in particular the natural signal sequence of the secreted protein, if the latter is functional in a hepatic cell. It may also be any appropriate heterologous sequence. By way of example, there may be mentioned the signal sequence of apolipoprotein AI. In addition, the cassette generally comprises a region situated in 3′ which specifies a signal for termination of transcription and a polyadenylation signal. The SV40 virus polyA site may be used for example. It is understood that the choice of these signals is within the general capabilities of persons skilled in the art.

The invention also relates to a pharmaceutical composition comprising a vector as described above. The pharmaceutical compositions of the invention may be formulated for administration by the topical, oral, parenteral, intranasal, intravenous, intramuscular, subcutaneous, intraocular or transdermal route and the like.

Preferably, the pharmaceutical composition contains pharmaceutically acceptable vehicles for an injectable formulation. They may be in particular isotonic sterile saline solutions (monosodium or disodium phosphate, sodium, potassium, calcium or magnesium chloride and the like, or mixtures of such salts), or dry, particularly freeze-dried, compositions which, upon addition, depending on the case, of sterilized water or of physiological saline, allow the constitution of injectable solutions. Other excipients may be used, such as for example a hydrogel. This hydrogel may be prepared from any biocompatible and noncytotoxic (homo- or hetero-) polymer. Such polymers have, for example, been described in application WO 93/08845. Some of them, such as in particular those obtained from ethylene and/or propylene oxide are commercially available. The virus doses used for the injection may be adjusted according to various parameters, and in particular according to the mode of administration used, the relevant pathology, the gene to be expressed, or the desired duration of treatment. In general, the recombinant adenoviruses of the invention are formulated and administered in the form of doses of between 10⁴ and 10¹⁴ pfu, and preferably 10⁶ to 10¹⁰ pfu. The term pfu (“plaque forming unit”) corresponds to the infectivity of an adenovirus solution, and is determined by infecting an appropriate cell culture, and measuring, generally after 15 days, the number of plaques of infected cells. The techniques for determining the pfu titre of a viral solution are well documented in the literature.

Because of their hepatospecific character, the vectors (particularly adenoviruses) according to the invention can also be used for the creation of animal models of hepatic pathologies.

Moreover, the invention also relates to any cell modified by a vector (particularly an adenovirus) as described above. These cells can be used for the production of recombinant proteins in vitro. They may also be intended for implantation into an organism, according to the methodology described in application WO 95/14785. These cells are preferably hepatic cells.

The subject of the present invention is also a process for the production of recombinant proteins comprising the infection or transfection of a cell population with a vector, a recombinant adenovirus or the corresponding viral genome comprising an expression cassette encoding a desired protein, the culture of the said recombinant cell population, and the recovery of the said protein produced. Advantageously, for carrying out the process of the invention, cells of hepatic origin are used. They may be established lines or primary cultures.

The invention also relates to new variants of the promoter of the human apolipoprotein AII gene having improved expression characteristics. These variants according to the invention comprise, in particular, a repeat of J units as described above. In addition, these variants advantageously comprise a deletion in the region between residues −710 and −150 of the native promoter.

The invention also relates to the hepatospecific and inducible promoters derived from the promoter of the human apolipoprotein AII gene comprising a regulatory region composed of one or more J units of the apolipoprotein AII promoter and a hepatospecific promoter region derived from another promoter.

Advantageously, the hepatospecific promoter region is derived from a hepatospecific promoter other than the promoter of the human gene for apolipoprotein AII. Preferably, it is composed of a promoter chosen from the serum albumin promoter, the apolipoprotein AI promoter, the apolipoprotein Cs promoter, the apolipoprotein B100 promoter, the fibrinogen gamma chain promoter (JBC 270 (1995) 28350), the promoter of the gene for human phenylalanine hydroxylase (PNAS 93 (1996) 728), the promoter of the AMBP gene (NAR 23 (1995) 395), the promoter of the factor X gene (JBC 271 (1996) 2323), the cytochrome P450 1A1 promoter (PNAS 92 (1995) 11926), the hepatitis B virus promoter (Biol. Chem. 377 (1996) 187) or the a-antitrypsin promoter. The promoter region used preferably consists of the region which is necessary and sufficient for the hepatic expression (minimum promoter). This region generally comprises the TATA box, and may be prepared according to conventional molecular biology techniques, as indicated in the references cited. Thus, the first 209 base pairs of the promoter of the factor X gene are sufficient to confer hepatic expression (JBC cited above). Likewise, fragments −20 to −23; −54 to −57 and −66 to −77 of the fibrinogen gamma chain promoter constitute a minimum promoter allowing hepatospecific expression. These regions can be joined to the J regions (preferably 1 to 5) according to the methodology described above and illustrated in the examples, in order to generate hepatospecific and inducible promoters. In addition, these promoters may carry additional regulatory sequences of the “enhancer” type, which make it possible to enhance the levels of expression.

The hepatospecific promoter region may also be composed of a ubiquitous promoter coupled to an enhancer element conferring hepatospecific expression.

In this regard, the enhancer element conferring the hepatospecific character may be chosen from the enhancer of the apolipoproteins E/CI (J. Biol. Chem., 268 (1993) 8221-8229 and J. Biol. Chem., 270 (1995) 22577-22585), the albumin enhancer (Gene therapy, 3 (1996) 802-810), the transthyretin enhancer (Mol. Cell Biol. 15 (1995) 1364-1376), the hepatitis B virus enhancer (Biol. Chem. 377 (1996) 187) or artificial enhancers contained in the HNF (hepatic nuclear factors) site, sites for binding to the orphelin receptors, members of the steroid hormone receptors (Human gene therapy, 7 (1996) 159-171).

The ubiquitous promoter may be any promoter which is nonspecific for a tissue. It may be in particular a viral promoter or a housekeeping promoter. Among the viral promoters, there may be mentioned more particularly the SV40 promoter (Mol. Cell Biol. 1982; 2: 1044-1051); the RSV LTR (Rous sarcoma virus long terminal repeat) promoter (PNAS USA, 1982; 79: 6777-6781); the CMV (human cytomegalovirus) IE promoter (Gene 1986; 45: 101-105); the MOMLV (Moloney murine leukaemia virus) LTR promoter (Gene Therapy 1996; 3: 806-810) and the promoter of the HSV-TK (Thymidine Kinase) gene (Nucleic Acid Res 1980; 8: 5949-5964). Among the housekeeping promoters, there may be mentioned the promoter of the genes human EF-1alpha (elongation factor) (Gene 1993, 134: 307-308), chicken Beta-actin (Nucleic Acids Res. 1983; 11: 8287-8301), the POL II (mouse RNA polymerase II) promoter (Mol. Cell Biol. 1987; 7: 2012-2018); PGK (Phosphoglycerate Kinase) (Gene 1987; 61: 291-298); H4 Histone (Mol. Cell Biol. 1985; 5: 380-398), the HMG (human Hydroxymethylglutaryl CoA reductase) (Mol. Cell Biol. 1987; 7: 1881-1893), the HK2 (rat Hexokinase II) (J. Biol. Chem. 1995; 270: 16918-16925) and the PRP (Prion) (Virus genes 1992; 6: 343-356). Any other ubiquitous promoter known to persons skilled in the art can also be used.

The hepatospecific promoter region can be obtained by coupling, according to conventional molecular biology techniques, all or a functional part of a ubiquitous promoter with the above enhancer element. In particular, the oligonucleotides corresponding to the J sites containing bases −737 to −715 of the human apoAII promoter can be cloned into the BamHI/GglII sites of pIC20H (Gene 1984; 32: 481-485), digested with HindIII, and subcloned in 5′ of the chosen ubiquitous promoter, for example of the Thymidine Kinase (TK) promoter into the plasmid pBLCAT4 (Nucl. Acid Res. 1987; 15: 5490), to give a vector containing the J sites and a ubiquitous promoter in front of a gene of interest. The hepatic enhancer can be added either in 5′ of the promoter or in 3′ of the polyadenylation site.

These variants are particularly advantageous because they combine the properties of strength of expression, of tissue specificity and of inducibility. These various variants can be used for the expression of genes of interest, in vitro and in viva as indicated above and illustrated in the examples.

The invention also relates to recombinant vectors comprising an expression cassette composed of a gene of interest under the control of a promoter as described above.

The invention also relates to a composition comprising a recombinant vector as described above and an activator of PPAR, for use which is simultaneous or spread out over time.

The recombinant vector is advantageously a recombinant adenovirus as defined above, and the activator of PPAR is advantageously an activator of PPARα.

Among the activators of PPARα, there may be used more particularly fibrates as well as any compound increasing the expression of transcription factors binding to the J sites.

By way of preferred examples of fibrates, there may be mentioned, for example, fibric acid and analogues thereof such as in particular gemfibrozil (Atherosclerosis 114 (1) (1995) 61), bezafibrate (Hepatology 21 (1995) 1025), ciprofibrate (BCE&M 9(4) (1995) 825), clofibrate (Drug Safety 11 (1994) 301), fenofibrate (Fenofibrate Monograph, Oxford Clinical Communications, 1995), clinofibrate (Kidney International. 44(6) (1993) 1352), pirinixic acid (Wy-14,643) or 5,8,11,14-eicosatetranoic acid (ETYA). These various compounds are compatible with a biological and/or pharmacological use in vitro or in vivo.

By way of examples of compounds increasing the expression of transcription factors binding to the J sites, there may be mentioned in particular the retinoids, which activate the expression of RXR and HFN4.

Moreover, the compositions according to the invention may comprise several PPAR activators in combination, in particular a fibrate or a fibrate analogue combined with a retinoid.

As indicated above, the vector and activator can be used simultaneously or spaced out over time. In addition, they can be packaged separately. According to a preferred embodiment, the vector and the activator are packaged separately and used spaced out over time. In particular, the vector is advantageously used first, then, in a second stage, the PPAR activator. The term used designates the bringing of the said vector or activator into contact with the cells, in vitro, ex vivo or in vivo. In vitro or ex vivo, the bringing into contact can be carried out by incubating a cellular population as mentioned above with the vector (for example from 0.01 to 1000 μg of vector per 10⁶ cells, or of virus with a multiplicity of infection (MOI) of 0.1 to 1000), followed by incubation with the activator (generally in a concentration range of between 10-3 mM and 10 mM, preferably between 10 μM and 500 μM). In viva, for example for the creation of transgenic animals or for the hepatospecific expression of genes of interest, the bringing into contact generally comprises the administration of the vector (under the conditions described above) followed by the administration of the activator. In this regard, the activator can be administered by the oral route, for example in the diet (for animals in particular) or in the form of gelatin capsules (for man). The daily doses administered to animals are of the order of 0.01 to 1% (weight/weight), preferably from 0.2 to 0.5% (weight/weight). A typical daily dose in mice for example is 50 mg. A typical daily dose of fenofibrate in man varies between 100 and 300 mg, preferably around 200 mg, which corresponds to a plasma concentration of about 15 μg/ml (Vidal, 1996). In addition, repeated administrations/incubations of vector and/or of activator can be carried out.

The present invention will be described more fully with the aid of the following examples, which should be considered as illustrative and nonlimiting.

### LEGEND TO THE FIGURES

FIG. 1: Structure of the apoAII promoter and of the deleted forms.

FIG. 2: Strategy for duplication of the J region.

FIG. 3: Structure of the apoAII promoters comprising a duplication of the J region in 5′, optionally combined with internal deletions.

FIG. 4: Structure of the apoAII promoters comprising a duplication of the J region internally, optionally combined with internal deletions.

FIG. 5: Representation of a recombinant adenovirus.

FIG. 6: Inducibility and strength of the recombinant adenovirus in vivo.

### GENERAL MOLECULAR BIOLOGY TECHNIQUES

The methods conventionally used in molecular biology, such as preparative extractions of plasmid DNA, centrifugation of plasmid DNA in caesium chloride gradient, agarose or acrylamide gel electrophoresis, purification of DNA fragments by electroelution, phenol or phenol-chloroform extractions of proteins, ethanol or isopropanol precipitation of DNA in saline medium, transformation in Escherichia coli and the like, are well known to persons skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987].

The pBR322- and pUC-type plasmids and the phages of the M13 series are of commercial origin (Bethesda Research Laboratories). For the ligations, the DNA fragments can be separated according to their size by agarose or acrylamide gel electrophoresis, extracted with phenol or with a phenol/chloroform mixture, precipitated with ethanol and then incubated in the presence of phage T4 DNA ligase (Biolabs) according to the supplier's recommendations. The protruding 5′ ends can be filled with the Klenow fragment of DNA Polymerase I of E. coli (Biolabs) according to the supplier's specifications. The protruding 3′ ends are destroyed in the presence of phage T4 DNA Polymerase (Biolabs) used according to the manufacturer's recommendations. The protruding 5′ ends are destroyed by controlled treatment with S1 nuclease.

Site-directed mutagenesis in vitro by synthetic oligodeoxynucleotides can be carried out according to the method developed by Taylor et al. [Nucleic Acids Res. 13 (1985) 8749-8763], using the kit distributed by Amersham. Enzymatic amplification of DNA fragments by the so-called PCR technique [Polymerase-catalyzed Chain Reaction, Saiki R. K. et al., Science 230 (1985) 1350-1354; Mullis K. B. and Faloona F. A., Meth. Enzym. 155 (1987) 335-350] can be carried out using a “DNA thermal cycler” (Perkin Elmer Cetus) according to the manufacturer's specifications. The nucleotide sequences can be checked by the method developed by Sanger et al. [Proc. Natl. Acad. Sci. USA, 74 (1977) 5463-5467], using the kit distributed by Amersham.

### EXAMPLES

### Example 1

### Cloning of the Promoter of the Human Gene for Apolipoprotein AII

This example describes the cloning of the promoter of the human gene for apoAII. It is understood that any other technique can be used to reclone this promoter. Moreover, it is also possible, using cloned fragments, to prepare according to conventional molecular biological techniques versions which are shorter in 5′ and/or in 3′.

1.1. Cloning a −911/+29 Fragment

The human apolipoprotein AII promoter was cloned by PCR using a human genomic DNA. Primers ATC GAA GCT TCT GAT ATC TAT TTA ACT GAT (SEQ ID No. 3) and CGT CTC TGT CCT TGG TGT CTG GAT CCA TCG (SEQ ID No. 4) which introduce, for the first, an HindIII site in 5′ of the promoter, and for the second, a BamHI site in 3′, made it possible to clone the promoter from position −911 to position +29. The sequence of the promoter was confirmed by sequencing (SEQ ID No. 1) and the HindIII-BamHI fragment introduced into the vector pBLCAT5 for verification of the transcriptional activity.

1.2. Cloning of a −911/+160 Fragment

A fragment comprising the apoAII promoter of residues −911/+160 was also obtained from the genomic library, and then cloned into the vector pBLCAT5.

### Example 2

### Construction of Variants of the AII Promoter

2.1. Creation of Truncated Forms

This example describes the construction of variants of the apoAII promoter comprising an internal deletion, in the region between the regulatory elements located at the level of nucleotides −903 to −720, and −126 to −33. These variants were constructed from the fragments −911/+29 and −911/+160 described in Example 1.

Using the vectors pBLCAT5 comprising the complete promoter in the form of a −911/+29 or −911/+160 fragment, the −210 to +29 or −210 to +160 region was obtained by PCR using, as primer, an oligonucleotide −210/−198 of sequence 5′-GACTCTAGATGTACCCCCTTA-3′ (SEQ ID No. 5) and an oligonucleotide internal to the CAT gene. The fragment obtained was cloned into a plasmid pBLCAT5 to give the plasmids −210/+160AII-CAT and −210/+29AII-CAT. The distal region −911 to −653 (N-1) was obtained by digesting the fragment −911 to +29 by means of the AluI enzyme, and then cloning of the fragment obtained into the plasmids −210/+160AII-CAT and −210/+29AII-CAT. The distal region −911 to −708 (N-J) was obtained by PCR using, as primer, an oligonucleotide −708/−722 of sequence 5′-GGAAGCTGCAGAGGCTTCTACCAG-3′ (SEQ ID No. 6). The fragment obtained was then cloned into the plasmids −210/+160AII-CAT and −210/+29AII-CAT.

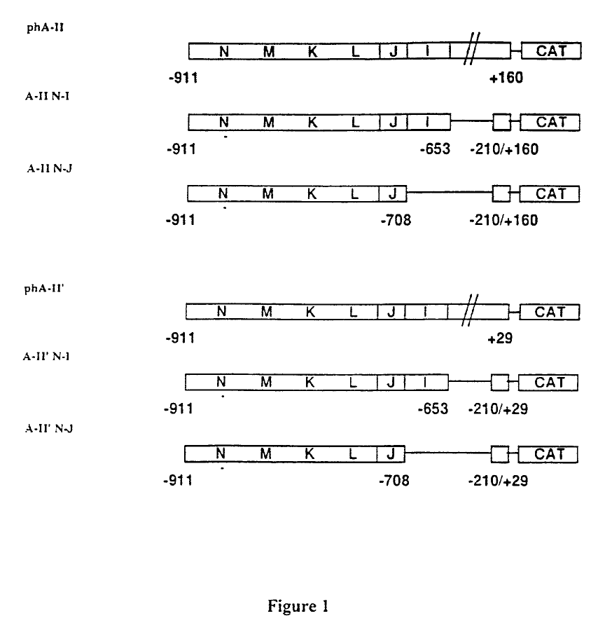

The structure of the promoters is represented in FIG. 1.

2.2. Duplication of the J Site

This example describes the construction of variants of the AII Promoter in which the J region has been repeated.

The following two oligonucleotides, corresponding to the J site, were synthesized using a DNA synthesizer.

| | | |
|---|---|---|
| Oligo 1: | 5′-gatcctTCAACCTTTACCCTGGTAGa-3′ | (SEQ ID |
| | No. 7) | |
| Oligo 2: | 5′-gatctCTACCAGGGTAAAGGTTGAag-3′ | (SEQ ID |
| | No. 8) | |

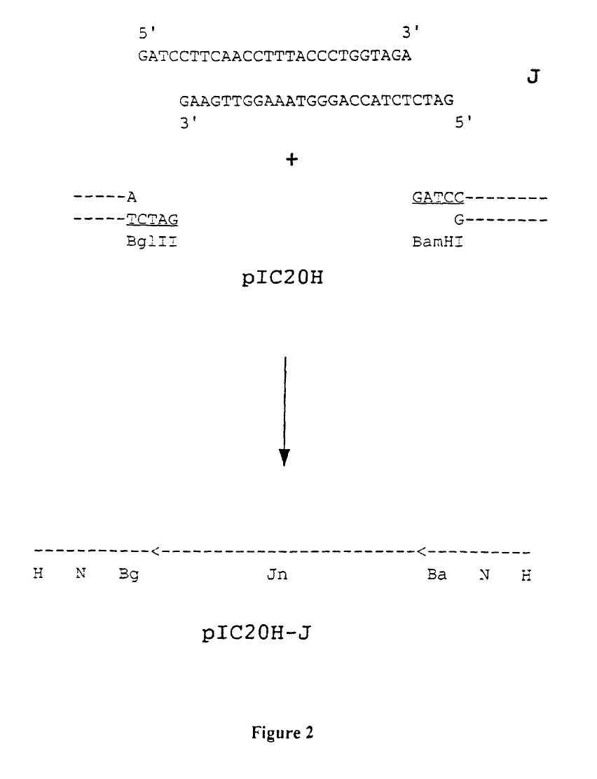

These oligonucleotides reconstitute, at the 3′ end, a BamHI site and at the 5′ end, a BglII site. These oligonucleotides were hybridized together and the fragment obtained was cloned at the BamHI-BglII sites into the vector pIC20H (FIG. 2). The resulting plasmids pIC20H-J were analysed to determine the copy number of J sites inserted, as well as their respective orientation.

The following plasmids were selected:

a plasmid carrying a single copy of the J site,

a plasmid carrying 2 copies of the J site in the same orientation,

a plasmid carrying 2 copies of the J site, in opposite orientation.

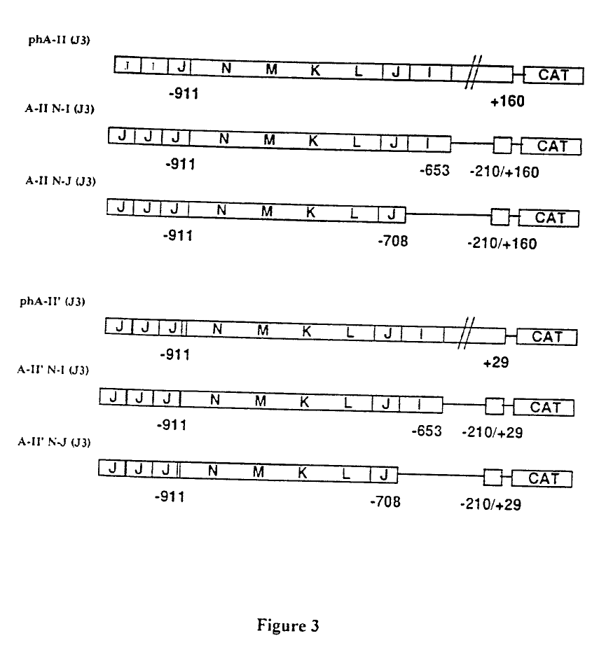

To construct the variants for the apoAII promoter comprising J units repeated in 5′, the inserts contained in the above plasmids were excised in the form of HindIII fragments and cloned into 5′ of the apoAII promoter (Example 1) or variants described in Example 2.1, at the level of a HindIII site. The structure of the resulting promoters is given in FIG. 3.

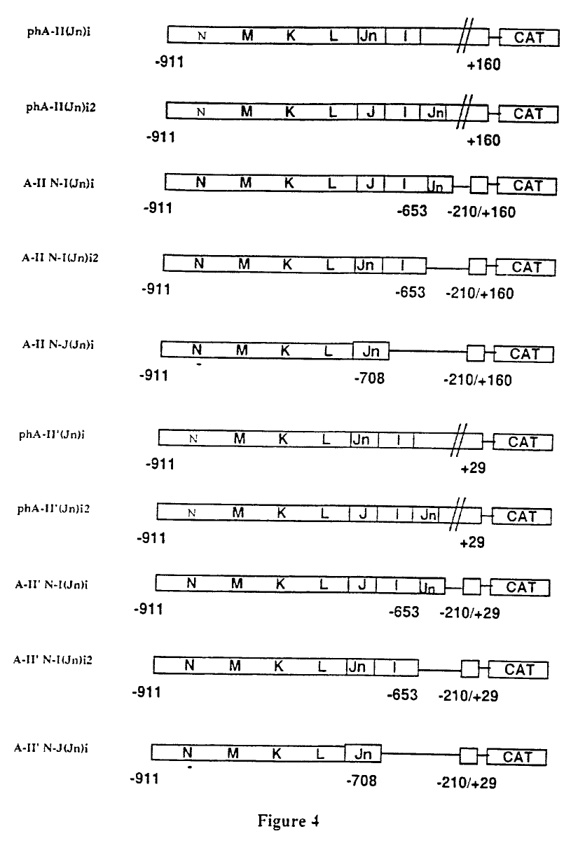

To construct the variants of the apoAII promoter comprising J units repeated internally, the inserts contained in the above plasmids were excised in the form of appropriate restriction fragments, and then cloned into the apoAII promoter (Example 1) or into the variants described in Example 2.1, at the level of a corresponding site situated between the native J region and the regulatory region −126-33 of the native promoter. The structure of the resulting promoters is given in FIG. 4.

The copy number of the J region in the final construct is determined by the choice of the plasmid.

### Example 3

### Study of the Functionality of the Variants of the apoAII Promoter

The functionality of the promoters was studied by transfection into a hepatic cell line, the HepG2 cells. A control experiment was carried out in a nonhepatic cell line, the Hela cells.

The transfections into the HepG2 cells were performed at 50-60% confluence by the calcium phosphate precipitation method. The cells were cotransfected with a mixture of plasmids comprising:

the test plasmid

a plasmid for expression of PPARA (PBK-CMV-PPARα) or the corresponding empty plasmid (PBK-CMV), and

0.5 μg of a plasmid for expression of β-Gal (CMV-β-Gal) as control of the efficiency of transfection.

All the transfections were performed with the same quantity of total DNA. 4 hours after the transfection, the cells were washed in PBS, and then incubated for 24 hours with the fibrate (Wy-14643, 1 μM). The CAT activity was then determined according to the method of Gorman et al., (Mol. Cell. Biol. 2 (1982) 1044). The results are then expressed in terms of the efficiency of transfection as measured by the expression of β-Gal.

The results obtained for two series of experiments are presented in Tables 1 and 2 below.

TABLE 1
Activity of the promoters on hepatic cells

| | | | CAT | β-Gal |
|---|---|---|---|---|
| Promoter | Vector | Activator | activity | correction |
| phA-II | PBK-CMV | — | 2.52 | 5.52 |
| | PBK-CMV | Wy | 5.37 | 11.75 |
| | mPPARa | — | 12.44 | 27.22 |
| | mPPARa | Wy | 12.04 | 26.34 |
| phA-II(J3) | PBK-CMV | — | 4.17 | 9.13 |
| | PBK-CMV | Wy | 7.80 | 17.06 |
| | mPPARa | — | 15.67 | 34.29 |
| | mPPARa | Wy | 24.96 | 54.61 |
| phA-II N-I | PBK-CMV | — | 1.31 | 2.86 |
| | PBK-CMV | Wy | 2.46 | 5.38 |
| | mPPARa | — | 3.58 | 7.83 |
| | mPPARa | Wy | 14.77 | 32.31 |
| phA-II N-I(J3) | PBK-CMV | — | 1.32 | 2.89 |
| | PBK-CMV | Wy | 2.95 | 6.45 |
| | mPPARa | — | 7.81 | 17.10 |
| | mPPARa | Wy | 17.51 | 38.32 |
| phA-II N-J | PBK-CMV | — | 0.37 | 0.80 |
| | PBK-CMV | Wy | 0.41 | 0.90 |
| | mPPARa | — | 0.44 | 0.97 |
| | mPPARa | Wy | 1.11 | 2.42 |
| phA-II N-J(J3) | PBK-CMV | — | 0.46 | 1.00 |
| | PBK-CMV | Wy | 0.77 | 1.69 |
| | mPPARa | — | 4.04 | 8.83 |
| | mPPARa | Wy | 13.06 | 28.59 |

TABLE 2
Activity of the promoters on hepatic cells

| | | | CAT | β-Gal |
|---|---|---|---|---|
| Promoter | Vector | Activator | activity | correction |
| phA-II | PBK-CMV | — | 5.03 | 2.12 |
| | PBK-CMV | Wy | 6.68 | 3.41 |
| | mPPARa | — | 49.77 | 16.99 |
| | mPPARa | Wy | 60.86 | 32.20 |
| phA-II(J3) | PBK-CMV | — | 5.80 | 2.16 |
| | PBK-CMV | Wy | 8.17 | 3.50 |
| | mPPARa | — | 69.71 | 17.08 |
| | mPPARa | Wy | 81.20 | 21.15 |
| phA-II N-I | PBK-CMV | — | 2.91 | 1.25 |
| | PBK-CMV | Wy | 1.35 | 0.67 |
| | mPPARa | — | 20.85 | 3.88 |
| | mPPARa | Wy | 32.34 | 8.86 |
| phA-II N-I(J3) | PBK-CMV | — | 2.20 | 1.19 |
| | PBK-CMV | Wy | 3.01 | 1.68 |
| | mPPARa | — | 57.07 | 11.65 |
| | mPPARa | Wy | 75.39 | 17.53 |
| phA-II N-J | PBK-CMV | — | 2.02 | 1.50 |
| | PBK-CMV | Wy | 0.56 | 0.37 |
| | mPPARa | — | 1.65 | 0.49 |
| | mPPARa | Wy | 2.91 | 1.26 |
| phA-II N-J(J3) | PBK-CMV | — | 1.04 | 0.64 |
| | PBK-CMV | Wy | 0.55 | 0.60 |
| | mPPARa | — | 21.40 | 3.87 |
| | mPPARa | Wy | 30.56 | 9.95 |

These results show clearly that the duplication of the J unit in the promoter does not alter the strength of the promoter, and very significantly increases its inducibility by the fibrates.

Moreover, in a control experiment carried out in Hela cells, no inducibility by the fibrates or by PPAR was observed. This demonstrates that the promoters according to the invention conserve their tissue specificity.

These results therefore demonstrate that the promoters according to the invention are strong, highly inducible, and specific for hepatic cells. In addition, the promoters phA-II N-I(J3); phA-II′ N-I(J3); phA-II N-J(J3); phA-II′ N-J(J3); phA-II N-I (J3i); phA-II′ N-I(J3i); phA-II N-J(J3i) and phA-II′ N-J(J3i) possess the advantage of being small in size, compared with the native apoAII promoter. This therefore makes it possible advantageously, in a gene transfer and/or expression vector, to have a higher cloning capacity.

### Example 4

### Construction of Inducible and Hepatospecific Adenoviruses

This example describes the construction of inducible and hepatospecific adenoviruses giving very high levels of expression. These adenoviruses are useful for the expression of genes in vitro, ex vivo or in vivo. The adenoviruses described were constructed from the Ad5 serotype. It is understood that any other serotype can be used, and in particular the serotypes Ad2, Ad7, Ad12 and CAV2. The adenoviruses were constructed by homologous recombination, in a packaging line, between a shuttle vector providing the left part of the viral genome and the linearized adenovirus DNA, providing the right part of the viral genome.

4.1. Construction of the Shuttle Vectors

The shuttle vector constructed carries an expression cassette consisting of an apoAII promoter and a nucleic acid encoding a secreted molecule: apolipoprotein AI (apoAI). This vector provides, in addition, the left part of the viral genome, that is to say the left ITR and a region allowing recombination.

The shuttle vectors serving for the construction of the adenovirus were constructed from a plasmid pCO5 (WO 96/22378), from a plasmid pIC20H-Alb-U-AI-SV40 which contains the first intron of apoAI and the cDNA for apoAI under the control of a rat albumin promoter, and from a plasmid pBLAIICAT5 which contains an apoAII promoter as described in Example 1 or a variant according to Example 2.

a) Construction of plC20H-Alb-U-AI-SV40

The two primers GCG GCC GCT TCG AGC AGA CAT GAT AA (SEQ ID No. 9) and CGA TCT CAA GGG CAT CGG TCG ACG G (SEQ ID No. 10) allowed the amplification of the SV40 polyadenylation sequences in the late orientation. The 5′ primer introduces an NotI site upstream of this sequence and the 3′ primer introduces a semi-NruI site. The PCR fragment obtained is treated with klenow so as to obtain blunt ends and cloned into the vector pIC20H cleaved with SmaI and NruI, in the orientation which regenerates an NruI site. The resultant plasmid is called pIC2OH-SV40.

The construct pXL2336 containing an apolipoprotein AI minigene has been described previously (WO 94/25073). A PCR fragment is amplified from pXL2336 by means of the primers GGG ATC CGC TGG CTG CTT AGA GAC TGC (SEQ ID No. 11) and GGC GGC CGC CGG GAA GGG GGG CGG CGG (SEQ ID No. 12) which introduce respectively a BamHI site upstream of the first exon of ApoAI and an NotI site downstream of the coding sequence of ApoAI.

The PCR fragment is cloned into pCRII (Invitrogen) and its sequence checked. The BamHI/NotI fragment containing the cDNA and the first intron of apoAI is then cloned at the same sites of the plasmid pIC2OH-SV40 to generate the plasmid pIC2OH-AI-SV40.

The oligonucleotides CAC GTG CTT GTT CTT TTT GCA GAA GCT CAG AAT AAA CGC TCA ACT GTG GC (SEQ ID No. 13) and CGT GGC CAC AGT TGA GCG TTT ATT CTG AGC TTC TGC AAA AAC AAG AAG CA (SEQ ID No. 14) are then hybridized with each other and cloned at the DsaI site of pIC20H-AI-SV40 such that the PmlI site created during this cloning is on the intron side and that the DsaI site is regenerated at the other end. These oligonucleotides make it possible to introduce a fragment of the untranslated 5′ part of the messenger RNA for β-globin. The plasmid obtained is called pIC2OH-U-AI-SV40.

Finally, the HindIII/BglII fragment of the rat albumin promoter described by F. Troche et al. (Mol. Cel. Biol., 1989, 4759-4766) treated with klenow was cloned in the appropriate orientation into the plasmid pIC20H-U-AI-SV40 cleaved with BamHI and also treated with klenow to generate the plasmid pIC2OH-Alb-U-AI-SV40.

b) Construction of the Shuttle Vectors

The oligonucleotides

| | |
|---|---|
| CGT GGC AGG CAG CAG GAC GCA CCT CCT TCT | (SEQ ID No. 15) and |
| CGC AGT CTC TAA GCA GCC TTC GAA GCA TG | |
| CTT CGA AGG CTG CTT AGA GAC TGC GAG | (SEQ ID No. 16) |
| AAG GAG GTG CGT CCT GCT GCC TGC CA | |

are hybridized with each other, creating a cohesive SphI and a cohesive HpaII end. This fragment is introduced in a three-partner ligation with a HpaII/DraIII fragment (476/1518) of the plasmid pIC20HAlb-U-AI-SV40 containing the cDNA for ApoAI and a DraIII/SphI fragment (1518/239) of the same plasmid. The resulting plasmid is called pXL2699. This construct creates a BstBI site compatible with ClaI upstream of the cDNA fragment+intron of apoAI. The BstBI/SalI fragment of pXL2699, containing the ApoAI cDNA, is cloned into the plasmid pCO5 cleaved with ClaI and SalI. The resulting plasmid is called pCO5-U-AI-SV40.

The apoAII promoter selected (complete promoter or variants) is excised from the corresponding plasmid pBLAIICAT5 in the form of a HindIII-BamHI fragment, cloned after treating with klenov at the EcoRV site of the plasmid pCO5-U-AI-SV40 to generate the shuttle vectors pXLPromAII/AI. The following vectors are thus obtained:

| Shuttle vector | Version of the promoter |
|---|---|
| pXLAII/AI | −911/+29 |
| pXLhAII/AI | −911/+160 |
| pXLAII(J3)/AI | −911/+29, 3 units J in 5′ |
| pXLAIIN-I/AI | −911/−653; −210/+29 |
| pXLAIIN-I(J3)/AI | −911/−653; −210/+29; 3 units J in 5′ |
| pXLAIIN-J/AI | −911/−708; −210/+29 |
| pXLAIIN-J(J3)/AI | −911/−708; −210/+29; 3 units J in 5′ |

These vectors are validated for the expression of human apoAI by transient transfection into the 293 or Cos1 lines. It is understood that the nucleic acid encoding human apolipoprotein AI can be easily replaced, in the shuttle vectors, by any other nucleic acid encoding a molecule of interest.

4.2. Construction of the Adenoviruses

The adenoviruses were produced by homologous recombination, after cotransfection, in the appropriate cells, of two DNA fragments, one providing the left part of the genome of the recombinant virus (shuttle vector described in Example 4.1., possessing a deletion in the E1 region), the other providing the right part of the genome of the recombinant virus (optionally possessing a deletion in the E4 and/or E3 region).

a) Construction of Adenoviruses Defective for the E1 Region

The adenovirus Ad-AII/AI was obtained by homologous recombination in vivo between the DNA of the Ad-RSV-βGal virus and the shuttle vector pXL AII/AI, according to the following protocol; the shuttle vector pXL AII/AI linearized with BstXI and the DNA of the Ad-RSV-βGal virus linearized with the enzyme ClaI, were cotransfected into the 293 line in the presence of calcium phosphate, to allow the homologous recombination. The recombinant adenoviruses generated were then selected by plaque purification. After isolation, the recombinant adenovirus DNA was amplified in the 293 cell line, which gives a culture supernatant containing the unpurified recombinant defective adenovirus having a titer of about 10¹⁰ pfu/ml.

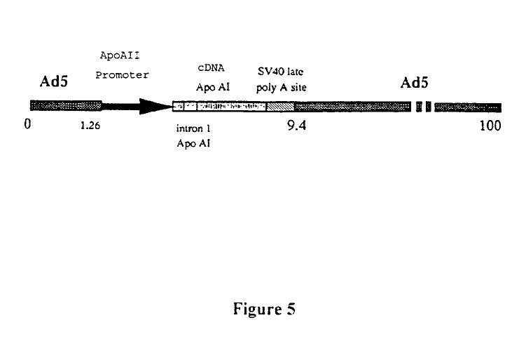

The viral particles are then purified by cesium chloride gradient centrifugation according to known techniques (see in particular Graham et al., Virology 52 (1973) 456), or by chromatography (FR 96 08164). The adenovirus Ad-AII/AI can be stored at −80° C. in 20% glycerol. The structure of the recombinant genome is presented in FIG. 5.

The same strategy is followed to construct the adenoviruses carrying various forms of AII promoters, starting with the shuttle vectors described in Example 4.1.

b) Construction of Adenoviruses Defective for the E1 and E4 Regions

IGRP2 cells (Yeh et al., J. Virol 70 (1996) 559), capable of transcomplementing the adenovirus E1 and E4 functions, are cotransfected with 5 mg of the shuttle vector digested with BstXI, and 10 mg of the virus DNA providing the functional deletion of the E4 region (for example Ad2dl808, Ad5dl1004, Ad5dl1007 or Ad5dl1014) digested with the enzyme ClaI. After the appearance of the cytopathic effect, the viruses are purified by at least two consecutive cycles of plating on a solid for the formation of plaques on IGRP2. The plaques corresponding to the infection of the desired virus (analysis of the DNA demonstrating the double deletion E1 and E4) are then amplified by consecutive cycles of infection. Stocks with a high titer are prepared by cesium chloride gradient purification. The viruses are stored at −80° C. according to the conventional techniques of persons skilled in the art.

### Example 5

### In vivo Activity of the Recombinant Adenoviruses

This examples describes the functional properties of the adenoviruses of the invention, after administration in vivo.

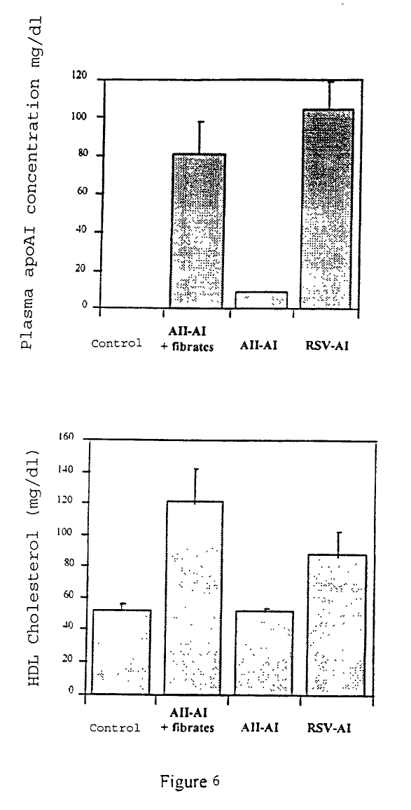

5×10⁹ pfu of the viruses Ad(ApoAIIprom-ApoAI-SV40 and Ad(RSV-ApoAI-bGH) were injected respectively into 8 and 3 C57b16 mice. Four of the mice injected with Ad(ApoAIIprom-ApoAI-SV40) were fed with fenofibrate at the dose of 0.5% (w/w) mixed with their food. The plasma concentrations of human apoAI and of HDL cholesterol were measured every week. The results obtained are presented in FIG. 6.

The plasma apoAI levels are below the detection threshold (10 mg/dl) for the mice injected with the Ad(ApoAIIprom-ApoAI-SV40) virus, 81±0.17 mg/dl for the fenofibrate-treated mice injected with the Ad(ApoAIIprom-ApoAI-SV40) virus and 84±15 mg/dl for the mice injected with the Ad(RSV-ApoAI-bGH) virus, which shows at least an 8-fold induction of the apoAII promoter under these conditions.

The HDL cholesterol level is not modified for the mice injected with the Ad(ApoAIIprom-ApoAI-SV40) virus (52±2 mg/dl): level identical to untreated animals. On the other hand, the HDL cholesterol level is increased on day 7 up to 88±14 mg/dl and 121±22 mg/dl respectively for the mice injected with the Ad(RSV-ApoAI-bGH) virus and the fenofibrate-treated mice injected with the Ad(ApoAIIprom-ApoAI-SV40) virus (FIG. 6).

These results demonstrate in vivo the strong, inducible and hepatospecific character of the promoters of the invention in an adenoviral context. Combined with the remarkable gene transfer properties of adenoviruses, the vectors of the invention exhibit highly advantageous performances for the transfer and expression of genes.

1. Recombinant vector for the inducible and hepatospecific expression of a molecule, characterized in that it comprises an expression cassette consisting of a nucleic acid encoding the said molecule, placed under the control of the promoter of the human apolipoprotein AII gene.

2. Recombinant vector according to claim 1, characterized in that the promoter comprises the regulatory elements located at the level of nucleotides −903 to −680; −573 to −255 and −126 to −33 of the human apolipoprotein AII gene promoter.

3. Recombinant vector according to claim 1 or 2, characterized in that the 3′ end of the promoter is between residues +5 and +35, more preferably +10 and +30 of the apoAII gene.

4. Recombinant vector according to one of claims 1 to 3, characterized in that the 5′ end of the promoter is between residues −950 to −905, preferably −925 to −910 of the apoAII gene.

5. Recombinant vector according to claim 1, characterized in that the promoter comprises the sequence SEQ ID No. 1 (residues −911 to +29).

6. Recombinant vector according to one of claims 1, 3 or 4, characterized in that the promoter comprises the regulatory elements located at the level of nucleotides −903 to −680, or −903 to −720, and −126 to −33 of the promoter of the human apolipoprotein AII gene, but not the intermediate elements located at the level of nucleotides −573 to −255.

7. Recombinant vector according to claim 6, characterized in that the promoter comprises a deletion in the region between residues −670 and −210, preferably a deletion of residues −653−210.

8. Recombinant vector according to claim 6, characterized in that the promoter comprises a deletion in the region between residues −710 and −150.

9. Recombinant vector according to claim 8, characterized in that the promoter comprises a deletion of residues −708−210.

10. Recombinant vector according to any one of the preceding claims, characterized in that the promoter comprises a repeat of J units.

11. Recombinant vector according to claim 10, characterized in that the promoter comprises from 2 to 5 J units.

12. Recombinant vector according to claim 10, characterized in that the repeat of J units is positioned in 5′ of the promoter.

13. Recombinant vector according to claim 10, characterized in that the repeat of J units is positioned in 3′ of the promoter.

14. Recombinant vector according to claim 10, characterized in that the repeat of J units is inserted into the sequence of the promoter.

15. Recombinant vector according to claim 10, characterized in that the promoter comprises a regulatory region composed of one or more J units and a hepatospecific promoter region.

16. Recombinant vector according to claim 15, characterized in that the hepatospecific promoter region is composed of a hepatospecific promoter chosen from the serum albumin promoter, the apolipoprotein AI promoter, the apolipoprotein Cs promoter, the apolipoprotein B100 promoter, the fibrinogen gamma chain promoter, the promoter of the gene for human phenylalanine hydroxylase, the promoter of the AMBP gene, the promoter of the factor X gene and the a-antitrypsin promoter.

17. Recombinant vector according to any one of claims 1 to 16, characterized in that it is a recombinant adenovirus.

18. Recombinant adenovirus according to claim 17, characterized in that it comprises a deletion in the E1 region of its genome.

19. Recombinant adenovirus according to claim 18, characterized in that it comprises a deletion of its E1a and E1b regions.

20. Recombinant adenovirus according to claim 18, characterized in that it comprises, in addition, a deletion in the E4 region of its genome.

21. Recombinant adenovirus according to claim 20, characterized in that the deletion in the E4 region affects all the open reading frames.

22. Recombinant adenovirus according to claims 18 to 21, characterized in that the expression cassette is inserted at the level of the E1 region, as a replacement for the deleted sequences.

23. Recombinant adenovirus according to claims 18 to 21, characterized in that the expression cassette is inserted at the level of the E4 region, as a replacement for the deleted sequences.

24. Recombinant adenovirus according to claims 18 to 21, characterized in that the expression cassette is inserted at the level of the E3 region.

25. Recombinant vector according to claim 1, characterized in that the molecule is a therapeutic protein.

26. Recombinant vector according to claim 25, characterized in that the therapeutic molecule is a protein secreted into the blood stream.

27. Recombinant vector according to claim 25, characterized in that the therapeutic protein is chosen from hormones, lymphokins, growth factors, neurotransmitters or precursors thereof or synthesis enzymes, trophic factors, apolipoproteins, tumour suppressors and the factors involved in clotting.

28. Adenoviral vector comprising an expression cassette consisting of a nucleic acid encoding a molecule of interest placed under the control of the promoter of the human apolipoprotein AII gene.

29. Variant of the promoter of the human apolipoprotein AII gene comprising a repeat of J units.

30. Variant according to claim 29, characterized in that it comprises from 2 to 5 J units.

31. Variant according to claim 29 or 30, characterized in that the additional J units are positioned in 5′ of the promoter.

32. Variant according to one of claims 29 to 31, characterized in that it comprises, in addition, a deletion in the region between residues −710 and −150 of the native promoter.

33. Variant of the promoter of the human apolipoprotein AII gene, characterized in that it comprises a regulatory region composed of one or more J units of the apolipoprotein AII promoter and a hepatospecific promoter region derived from another promoter.

34. Variant according to claim 33, characterized in that the hepatospecific promoter region is composed of a hepatospecific promoter other than the promoter of the human apolipoprotein AII gene.

35. Variant according to claim 33, characterized in that the hepatospecific promoter region is composed of a ubiquitous promoter coupled to an enhancer element conferring hepatospecific expression.

36. Cell modified by a vector according to one of claims 1 to 28.

37. Pharmaceutical composition comprising an adenovirus according to claim 17 and a pharmaceutically acceptable vehicle.

38. Process for the production of a desired recombinant protein comprising:
the infection or transfection of a cell population with a recombinant vector according to claim 1 or a viral genome comprising an expression cassette encoding the said desired protein,
the culture of the said recombinant cell population, and,
the recovery of the said protein produced.

39. Use of an adenovirus according to claim 17 for preparing a transgenic nonhuman animal model.

40. Composition comprising a recombinant vector according to one of claims 1 to 28 and an activator of PPAR, for a use which is simultaneous or spread out over time.

41. Composition according to claim 40, characterized in that the vector is a recombinant adenovirus according to claim 17.

42. Composition according to claim 40 or 41, characterized in that the activator of PPAR is an activator of PPARA.

43. Composition according to claim 42, characterized in that the activator of PPARA is chosen from fibrates and compounds increasing the expression of transcription factors binding to the J sites.

44. Composition according to claim 43, characterized in that the fibrate is chosen from fibric acid, gemfibrozil, benzafibrate, ciprofibrate, clofibrate, fenofibrate and clinofibrate.
